(19) United States
(12) Patent Application Publication
(10) Pub. No.: US 2026/0308215 A1
(43) Pub. Date: Oct. 8, 2026

(54) NOVEL BAX VARIANT AND PHARMACEUTICAL COMPOSITION COMPRISING SAME

(71) Applicant: CHA UNIVERSITY INDUSTRY-ACADEMIC COOPERATION FOUNDATION, Pocheon-si, Gyeonggi-do (KR)

(72) Inventors: Kwang-Hyun BAEK, Seoul (KR); Hae-Seul CHOI, Seongnam-si, Gyeonggi-do (KR)

(73) Assignee: CHA UNIVERSITY INDUSTRY-ACADEMIC COOPERATION FOUNDATION, Pocheon-si, Gyeonggi-do (KR)

(21) Appl. No.: 19/100,808

(22) PCT Filed: Aug. 7, 2023
(86) PCT No.: PCT/KR2023/011541

§ 371 Date: Feb. 3, 2025

(30) Foreign Application Priority Data
Aug. 8, 2022 (KR) 10-2022-0098471

(51) Int. Cl. C07K 14/47; A61K 38/00; G01N 33/50

(52) U.S. Cl. CPC C07K 14/4747; G01N 33/5011; A61K 38/00

(57) ABSTRACT

The present invention provides: a Bax protein variant in which lysine at position 128, lysine at position 190, or lysines at positions 128 and 190 in the Bax protein is (are) substituted with arginine; a method for increasing the half-life of a Bax protein, the method comprising substituting lysine at position 128, lysine at position 190, or lysines at positions 128 and 190 in the Bax protein with arginine; a pharmaceutical composition comprising the Bax protein variant; and a method for screening for an anticancer substance. The Bax protein variant according to the present invention can inhibit a protein degradation mechanism through ubiquitination and exhibits a longer half-life than the wild-type Bax protein.

### TECHNICAL FIELD

The present invention relates to variants of the Bax protein, which is an apoptotic protein. More specifically, the present invention relates to a Bax protein variant in which one or more lysine residues of the Bax protein are substituted with arginine; a method for increasing the half-life of a Bax protein comprising substituting one or more lysine residues of the Bax protein with arginine; a pharmaceutical composition comprising the Bax protein variant; and a method for screening an anticancer substance.

### BACKGROUND ART

Apoptosis is programmed cell death and relies on extrinsic and intrinsic signaling pathways. The extrinsic pathway is initiated by death receptors on the cell surface, whereas the intrinsic pathway is initiated by stress signals such as UV damage, hypoxia, oxidative stress, and DNA damage. The stress signals induce an apoptotic pathway initiated by pro-apoptotic proteins at the mitochondrial level.

Bax protein is a pro-apoptotic protein belonging to the Bcl-2 family and is involved in apoptosis induction in mitochondria. Bax, which is inactive (non-apoptotic) in the cytoplasm, increases mitochondrial outer membrane permeability (MOMP) by initiating translocation of the mitochondrial outer membrane after oligomerization with Bak (Pena-Blanco, A. et al., Bax, Bak and beyond-mitochondrial performance in apoptosis. FEBS J 2018, 285, 416-431, doi: 10.1111/febs. 14186; Grosse, L. et al., Bax assembles into large ring-like structures remodeling the mitochondrial outer membrane in apoptosis. EMBO J 2016, 35, 402-413, doi: 10.15252/embj.201592789). Thereafter, the protein cytochrome C located in the inner membrane moves into the cytoplasm (Danial, N. N. et al., Cell death: critical control points. Cell 2004, 116, 205-219, doi: 10.1016/s0092-8674 (04) 00046-7). Cytochrome C plays a critical role in the activation of procaspase-9, and induces the activation of caspases-3, -6, and -7 and apoptosis (Slee, E. A. et al., Serial killers: ordering caspase activation events in apoptosis. Cell Death Differ 1999, 6, 1067-1074, doi: 10.1038/sj.cdd.4400601; Kuribayashi, et al., What are caspases 3 and 7 doing upstream of the mitochondria? Cancer Biol Ther 2006, 5, 763-765, doi: 10.4161/cbt.5.7.3228).

Thus, Bax protein is an initiator of the intrinsic apoptosis pathway, and the effective control of Bax may provide an effective approach to the control of apoptosis. The present inventors have found that deubiquitinating enzymes USP1, USP7, USP12, or USP49 specifically bind to the Bax protein, which is known to be involved in apoptosis (Korean Patent No. 10-2282107, etc.).

### DETAILED DESCRIPTION OF THE INVENTION

### Technical Problem

The present inventors performed various studies in order to elucidate the mechanism for ubiquitination of the Bax protein. As the results thereof, it has been found that the protein variants, in which the specific amino acid residue(s) of the Bax protein (i.e., lysines at positions 128 and/or 190 lysine of the Bax protein) were substituted with arginine, show inhibited ubiquitination and significantly increased half-life.

Therefore, it is an object of the present invention to provide a Bax protein variant.

It is another object of the present invention to provide a method for increasing the half-life of a Bax protein.

It is still another object of the present invention to provide a pharmaceutical composition comprising the Bax protein variant as an active ingredient.

It is still another object of the present invention to provide a method for screening a substance having an anticancer activity using the Bax protein variant.

### Technical Solution

In accordance with an aspect of the present invention, there is provided a Bax protein variant in which lysine at position 128; lysine at position 190; or lysines at positions 128 and 190 in the Bax protein consisting of the amino acid sequence of SEQ ID NO: 1 is (are) substituted with arginine. The Bax protein variant may be a variant consisting of the amino acid sequence of SEQ ID NO: 3, 5, or 6.

In accordance with another aspect of the present invention, there is provided a method for increasing the half-life of a Bax protein, the method comprising substituting lysine at position 128 with arginine; lysine at position 190 with arginine; or lysines at positions 128 and 190 with arginine, in the Bax protein consisting of the amino acid sequence of SEQ ID NO: 1.

In accordance with still another aspect of the present invention, there is provided a pharmaceutical composition for preventing or treating a solid cancer, comprising the Bax protein variant as an active ingredient. The solid cancer may be gastric cancer, hepatic cancer, lung cancer, breast cancer, ovarian cancer, colorectal cancer, or the like.

In accordance with still another aspect of the present invention, there is provided a method for screening a substance having an anticancer activity, comprising (i) treating cells expressing both a Bax protein variant in which lysine at position 128; lysine at position 190; or lysines at positions 128 and 190 in the Bax protein consisting of the amino acid sequence of SEQ ID NO: 1 is (are) substituted with arginine and a deubiquitinating enzyme USP1, USP7, USP12, or USP49 with a candidate substance, followed by culturing the cell; and (ii) measuring whether apoptosis of the cells cultured in step (i) occurred, and then selecting a substance inducing apoptosis of the cells.

### Advantageous Effects

The Bax protein variant according to the present invention can inhibit the proteolytic mechanism through ubiquitination; and shows a significantly increased half-life, compared to the wild-type Bax protein. When the wild-type Bax protein is converted into the Bax protein variant according to the present invention, the half-life of the Bax protein can be increased. And, the variant according to the present invention can be usefully applied to a targeted anticancer agent for the treatment of solid cancer, through effective apoptosis of cancer cells. In addition, the variant according to the present invention can be usefully used as a target for promoting apoptosis of cancer cells and developing therapeutics. That is, the screening method of the present invention can be usefully used in screening a substance having an anticancer activity which targets the interaction of deubiquitinating enzymes USP1, USP7, USP12, and USP49 with a Bax protein.

### BRIEF DESCRIPTION OF THE DRAWINGS

FIG. 1 shows the protein structure of Bax protein including predicted ubiquitination sites (K128, K189, K190) (indicated by arrows) using Bioinformatics and PyMol.

FIG. 2 shows the results of Western blotting (left panel) and the results of immunoprecipitation with the Myc antibody (right panel), after transfection of HeLa cells with the HA-labeled ubiquitination enzyme and the Myc-labeled wild-type Bax or the Myc-labeled Bax variants (Bax K128R, Bax K189R, or Bax K190R) were transfected into Hela cells.

FIG. 3 shows the results obtained by measuring the expression levels of Bax from 0 to 24 hours through Western blotting, after respectively transfecting the wild-type Bax or the Bax variants (Bax K128R or Bax K190R) into Hela cells and then treating with cycloheximide, a protein synthesis inhibitor.

FIG. 4 shows the results obtained by calculating the relative Bax expression levels from 0 to 24 hours from the results of FIG. 3.

FIGS. 5a to 5c show the results by observing the cells at 0, 24, and 48 hours, after transfecting Hela cells with a mock vector, wild-type Bax, Bax K128R, and Bax K190R, respectively.

### BEST MODE FOR CARRYING OUR THE INVENTION

In order to find the sites estimated to have a high probability of ubiquitination in Bax, we short-listed BDM PUB (http://bdmpub.biocuckoo.org/), jci-bioinfo (http://www.jci-bioinfo.cn), RUBI (http://old.protein.bio.unipd.it/rubi/) and Netchop (http://www.cbs.dtu.dk/services/NetChop) and finally selected the lysine residues on the Bax surface using the PyMol program (https://pymol.org/2/). Then, we performed structural analysis of the Bax protein from the Protein Data Bank (PDB, https://www.rcsb.org/); and confirmed that the selected lysines were located on the surface of the Bax protein and that the lysine residues at positions 128 and 190 may be related to ubiquitination of the Bax protein (i.e., they can bind to glycine at the C-terminal of ubiquitin).

From said results, the present inventors performed conservative amino acid substitutions for the lysine residues, i.e. substitution with arginine including a basic side chain. That is, the Bax variants in which lysines at positions 128, 189, and 190 were substituted with arginine, respectively [i.e., Bax K128R (protein of SEQ ID NO: 3), Bax K189R (protein of SEQ ID NO: 4), and Bax K190R (protein of SEQ ID NO: 5)] were prepared. Among the variants, it was confirmed that the Bax K128R (protein of SEQ ID NO: 3) and the Bax K190R (protein of SEQ ID NO: 5) showed significant inhibition of ubiquitination. And, it has been found that when the Bax K128R (protein of SEQ ID NO: 3) and Bax K190R (protein of SEQ ID NO: 5) were treated with cycloheximide, a protein synthesis inhibitor, they showed a higher half-life, compared to the wild-type Bax protein. Therefore, the variants can be usefully applied to a targeted anticancer agent for the treatment of solid cancer, through effective apoptosis of cancer cells.

The present invention provides a variant of the Bax protein consisting of the amino acid sequence of SEQ ID NO: 1. That is, the present invention provides a Bax protein variant in which lysine at position 128; lysine at position 190; or lysines at positions 128 and 190 in the Bax protein consisting of the amino acid sequence of SEQ ID NO: 1 is (are) substituted with arginine.

The amino acid sequence of the Bax protein and the nucleotide sequence encoding the same are known in the art. For example, the amino acid sequence of the Bax protein is as shown in SEQ ID NO: 1 and the nucleotide sequence encoding the same is as shown in SEQ ID NO: 2.

In an embodiment, the Bax protein variant according to the present invention may be a variant in which lysine at position 128 in the Bax protein consisting of the amino acid sequence of SEQ ID NO: 1 is substituted with arginine, i.e., the variant consisting of the amino acid sequence of SEQ ID NO: 3. In another embodiment, the Bax protein variant according to the present invention may be a variant in which lysine at position 190 in the Bax protein consisting of the amino acid sequence of SEQ ID NO: 1 is substituted with arginine, i.e., the variant consisting of the amino acid sequence of SEQ ID NO: 5. In still another embodiment, the Bax protein variant according to the present invention may be a variant in which lysines at positions 128 and 190 in the Bax protein consisting of the amino acid sequence of SEQ ID NO: 1 are substituted with arginine, i.e., the variant consisting of the amino acid sequence of SEQ ID NO: 6.

The Bax protein variant according to the present invention may be prepared by substituting lysine at position 128; lysine at position 190; or lysines at positions 128 and 190 with arginine, according to a method conventionally used in the field of biotechnology. For example, using a gene encoding the Bax protein (e.g., a gene having the nucleotide sequence of SEQ ID NO: 2) as a template, polymerase chain reactions may be carried out with the primer sets of SEQ ID NOs: 7 and 8 below, so as to obtain a variant in which lysine at position 128 is substituted with arginine. In addition, for example, using a gene encoding the Bax protein (e.g., a gene having the nucleotide sequence of SEQ ID NO: 2) as a template, polymerase chain reactions may be carried out with the primer sets of SEQ ID NOs: 11 and 12 below, so as to obtain a variant in which lysine at position 190 is substituted with arginine.

Therefore, the present invention provides a method for increasing the half-life of a Bax protein, the method comprising substituting lysine at position 128 with arginine; lysine at position 190 with arginine; or lysines at positions 128 and 190 with arginine, in the Bax protein consisting of the amino acid sequence of SEQ ID NO: 1.

In an embodiment, the method of the present invention may comprise substituting lysine at position 128 with arginine, in the Bax protein consisting of the amino acid sequence of SEQ ID NO: 1 (i.e., converting the Bax protein consisting of the amino acid sequence of SEQ ID NO: 1 to the Bax protein variant consisting of the amino acid sequence of SEQ ID NO: 3). In another embodiment, the method of the present invention may comprise substituting lysine at position 190 with arginine, in the Bax protein consisting of the amino acid sequence of SEQ ID NO: 1 (i.e., converting the Bax protein consisting of the amino acid sequence of SEQ ID NO: 1 to the Bax protein variant consisting of the amino acid sequence of SEQ ID NO: 5). In still another embodiment, the method of the present invention may comprise substituting lysines at positions 128 and 190 with arginine, in the Bax protein consisting of the amino acid sequence of SEQ ID NO: 1 (i.e., converting the Bax protein consisting of the amino acid sequence of SEQ ID NO: 1 to the Bax protein variant consisting of the amino acid sequence of SEQ ID NO: 6).

The variants according to the present invention can be usefully applied to a targeted anticancer agent for the treatment of solid cancer, through effective apoptosis of cancer cells. Therefore, the present invention provides a pharmaceutical composition for preventing or treating a solid cancer, comprising said Bax protein variant as an active ingredient. The solid cancer may be gastric cancer, hepatic cancer, lung cancer, breast cancer, ovarian cancer, colorectal cancer, or the like.

The pharmaceutical composition of the present invention may comprise said Bax protein variant as an active ingredient as described above and a pharmaceutically acceptable carrier. The pharmaceutical composition of the present invention may be formulated to an injection, a lyophilized injection preparation, etc., according to conventional pharmaceutical methods. The pharmaceutically acceptable carrier may include a sterile aqueous solution (e.g., saline), a non-aqueous solvent, etc. And, if desired, it may comprise appropriate stabilizers, isotonic agents, preservatives, etc.

The dosage of the Bax protein variant contained as an active ingredient in the pharmaceutical composition of the present invention varies depending on the patient's condition and weight, degree of disease, administration route and period, and may be appropriately selected by a person skilled in the art. For example, the Bax protein variant may be administered at a dosage of 0.1 to 1,000 mg/kg as a daily dosage, and the administration may be administered once or several times per day.

The expression level of Bax is associated with malignant transformation, tumor progression, and metastasis, and therefore low expression of Bax is considered as a negative factor for cancer diseases. Reduced levels of Bax degradation are seen in invasive prostate cancer. Therefore, the Bax protein variant according to the present invention can be usefully used in screening a substance having anticancer activity which targets the interaction between the Bax protein and deubiquitinating enzymes (e.g., USP1, USP7, USP12, and USP49).

That is, the present invention provides a method for screening a substance having an anticancer activity, comprising (i) treating cells expressing both a Bax protein variant in which lysine at position 128; lysine at position 190; or lysines at positions 128 and 190 in the Bax protein consisting of the amino acid sequence of SEQ ID NO: 1 is (are) substituted with arginine and a deubiquitinating enzyme USP1, USP7, USP12, or USP49 with a candidate substance, followed by culturing the cell; and (ii) measuring whether apoptosis of the cells cultured in step (i) occurred, and then selecting a substance inducing apoptosis of the cells.

In the screening method of the present invention, the Bax protein variant may be a variant consisting of the amino acid sequence of SEQ ID NO: 3, 5, or 6.

Hereinafter, the present invention will be described more specifically by the following examples. However, the following examples are provided only for illustrations and thus the present invention is not limited to or by them.

### Examples

### 1. Test Method

### (1) Selection of Ubiquitination Sites and Preparation of Variants

Bioinformatics analysis was performed using the following three websites (Table 1) to analyze the ubiquitination site candidates in the Bax protein, and the lysine residues on the Bax surface were finally analyzed using the PyMol program (https://pymol.org/2/). The analysis results showed that lysines at the positions 128, 189, and 190 were the most repetitive amino acid residues, i.e., the amino acid residues with the highest efficiency, and were selected as ubiquitination site candidates.

TABLE 1
Ubiquitination Prediction Sites

http://bdmpub.biocuckoo.org/
http://www.jci-bioinfo.cn, RUBI(http://old.protein.bio.unipd.it/rubi/
http://old.protein.bio.unipd.it/rubi/

Total RNA was extracted from the HeLa cell line and cDNA was synthesized therefrom. Then, the Bax cDNA band was detected using the primer set (SEQ ID NOs: 13 and 14) in Table 2 below. The Bax gene (the gene of SEQ ID NO: 2) was cloned into a pcDNA3.1-6myc expression vector. Using the primer sets in Table 2 below, the Bax variants were prepared by substituting lysine at the positions 128, 189, or 190 of the Bax protein with arginine, respectively. Specifically, using the wild-type Bax gene (the gene of SEQ ID NO: 2) as a template, polymerase chain reactions (PCRs) were performed with each primer set. For the Bax K128R variant, the PCR (12 cycles in total) was performed under the conditions of: 30 seconds at 95° C., 30 seconds at 60° C., and 5 minutes at 68° C. For the Bax K189R variant and the Bax K190R variant, the PCR (12 cycles in total) was performed under the conditions of: 30 seconds at 95° C., 30 seconds at 54.4° C., and 5 minutes at 68° C.

TABLE 2

| | SEQ ID | | |
|---|---|---|---|
| Variant | NO | Sequence | |
| Bax K128R | 7 | Forward | 5′- TGC ACC AGG GTG CCG GAA -3′ |
| | 8 | Reverse | 5′- TTC CGG CAC CCT GGT GCC -3′ |
| Bax K189R | 9 | Forward | 5′- ATC TGG AGG AAG ATG GGC -3′ |
| | 10 | Reverse | 5′- GCC CAT CTT CCT CCA GAT -3′ |
| Bax K190R | 11 | Forward | 5′- ATC TGG AAG AGG ATG GGC -3′ |
| | 12 | Reverse | 5′- GCC CAT CCT CTT CCA GAT -3′ |
| Bax (wild- | 13 | Forward | 5′- ATG GAC GGG TCC GGG GAG -3′ |
| type) | | | |
| | 14 | Reverse | 5′- TCA GCC CAT CTT CTT CCA -3′ |

### (2) Transfection

Hela cells were cultured in Dulbecco's Modified Eagle's Medium (DMEM, Gibco, Grand Island, NY, USA) containing 10% fetal bovine serum (FBS, Gibco, Grand Island, NY, USA) and 1% penicillin and streptomycin (Gibco, Grand Island, NY, USA) in a 5% CO₂ incubator. For transfection, 600 μL of NaCl, 14 μL of polyethylenimine reagent (PEI; Polysciences, Inc., Warrington, PA, USA), and 2 μg of wild-type or variant DNA of pcDNA3.1 6myc-Bax were mixed and reacted at room temperature for 15 minutes. The mixture was added to 6 mL of the culture medium containing HeLa cells (1×10⁶ cells) and then cultured at 37° C. for 48 hours.

### (3) Immunoprecipitation

Transfected cells were lysed in a lysis buffer (50 mM Tris-HCl [pH 7.5], 1 mM EDTA, 10% glycerol, 300 mM NaCl, and 1% Triton X-100) on ice for 20 min, and then centrifuged at 13,000 rpm for 20 min. The supernatant was taken and the antibody (Myc antibody) was added thereto. The mixture was reacted overnight at 4° C. and then the A/G PLUS agarose beads (Santa Cruz Biotechnology, Santa Cruz, CA, USA) were added thereto. The mixture was reacted for 2 hours on a 4° C. rotator. Samples were washed twice with a washing buffer (a lysis buffer containing PMSF and PIC, 1:100), boiled with 2×SDS protein loading buffer for 7 minutes, loaded onto an SDS-PAGE gel, and then subjected to Western blotting.

### (4) Western Blotting

Transfected cells were lysed in a lysis buffer (50 mM Tris-HCl [pH 7.5], 1 mM EDTA, 10% glycerol, 300 mM NaCl, and 1% Triton X-100) on ice for 20 min, and then centrifuged at 13,000 rpm for 20 min. The supernatant was taken and then boiled with 2×SDS protein loading buffer for 7 minutes. The sample was loaded onto an SDS-PAGE gel and transferred to a polyvinylidene fluoride (PVDF) microporous membrane (Millipore, Billerica, MA, USA). After blocking the membrane with bovine serum albumin (KE, AU) for 1 hour, the primary antibodies (HA antibody and Myc antibody) were reacted with the membrane at 4° C. overnight. The mixture was washed with TBS-T (20 mM Tris-HCl pH 7.4, 100 mM NaCl, 0.1% (v/v) Tween 20) and the mouse secondary antibody (SERACARE, Milford, MA, USA) (1:30,000, 1% Skim milk) was added thereto, followed by reacting at room temperature for 2 hours. Protein bands were detected using an ECL reagent solution (Young In Frontier, Seoul, Korea).

### (5) Antibodies

Myc antibodies for labelling the wild-type Bax or Bax variants were used by culturing 9E10 cells in Roswell Park Memorial Institute Medium (RPMI, Gibco, Grand Island, NY, USA) containing 10% FBS (Gibco, Grand Island, NY, USA) and 1% penicillin/streptomycin (Gibco, Grand Island, NY, USA) in a 5% CO₂ incubator and then filtering through a 0.22 UM filter. And, HA antibody for labelling ubiquitin enzyme was used by culturing 12CA5 cells in RPMI (Gibco, Grand Island, NY, USA) containing 10% FBS (Gibco, Grand Island, NY, USA) and 1% penicillin/streptomycin (Gibco, Grand Island, NY, USA) in a 5% CO₂ incubator and then filtering through a 0.22 μM filter.

### (6) Measuring Half-Life of the Proteins

In order to compare and measure the half-life of wild-type Bax protein and lysine-substituted Bax proteins, HeLa cells were transfected with the wild-type or lysine-substituted Myc-Bax protein and cycloheximide, a protein synthesis inhibitor, was treated at a concentration of 100 UM after 24 hours therefrom. The cells were harvested from 0 h to 24 h (0, 12, 18, and 24 h) and then subjected to Western blotting.

### (7) Confirmation of Apoptosis Capability

In order to determine whether the Bax variants having longer half-life possess apoptosis capability similar to that of the wild-type protein, Hela cells were transfected with a mock vector, wild-type Myc-Bax, variant Myc-Bax K128R, and variant Myc-Bax K190R and the resulting cells were observed under a microscope at 0, 24, and 48 hours.

### (8) Confirmation of the Results and Statistical Analysis

Densitometric analysis was performed using Image J (National Institutes of Health, Bethesda, MD, USA), and t-test was performed using GraphPad Prism version 5 (GraphPad Software, La Jolla, CA, USA).

### 2. Test Results and Discussion

### (1) Protein Structure of Bax Protein Including Predicted Ubiquitination Sites (K128, K189, K190)

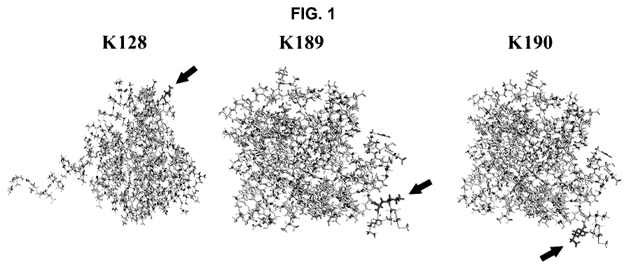

FIG. 1 shows the protein structure of Bax protein including predicted ubiquitination sites (K128, K189, K190) using Bioinformatics and PyMol, as described above.

### (2) Expression Analysis Through Western Blotting/Immunoprecipitation

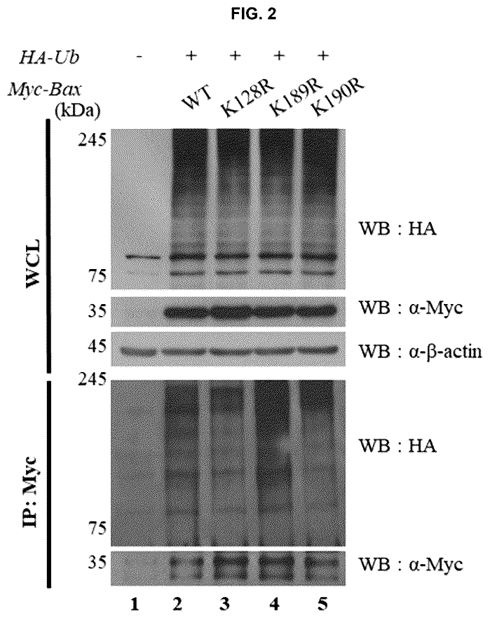

Western blotting was carried out after transfection of HeLa cells with the HA-labeled ubiquitination enzyme and the Myc-labeled wild-type Bax or the Myc-labeled Bax variants (Bax K128R, Bax K189R, or Bax K190R). The results thereof are shown in FIG. 2 (left panel). And, immunoprecipitation was carried out with the Myc antibody and the results thereof are shown in FIG. 2 (right panel). From the results in FIG. 2, it can be confirmed that ubiquitination of Bax K128R and Bax K190R is significantly inhibited.

### (3) Analysis of Bax Expression According to Treatment of Protein Synthesis Inhibitor

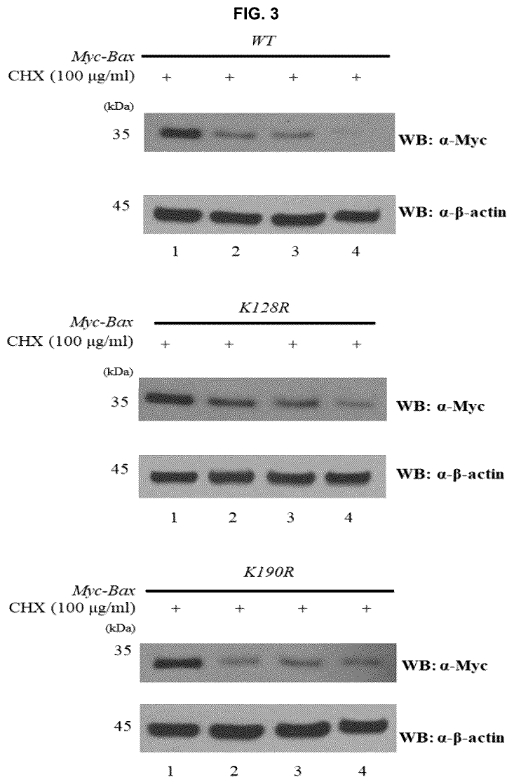

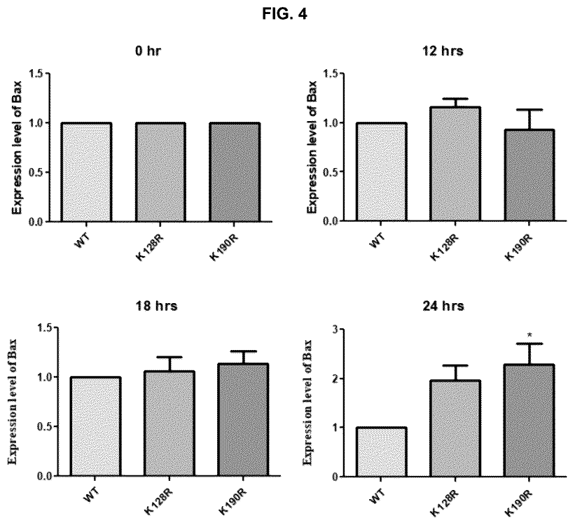

After respectively transfecting the wild-type Bax and the Bax variants (Bax K128R and Bax K190R) into Hela cells and then treating with cycloheximide, a protein synthesis inhibitor, the expression levels of Bax were measured from 0 to 24 hours through Western blotting. The results thereof are shown in FIG. 3. And, FIG. 4 shows the results obtained by calculating the relative Bax expression levels from 0 to 24 hours from the results of FIG. 3. As can be seen from the results in FIG. 3 and FIG. 4, when the expression level of the wild-type Bax at 24 hours is considered as 1, the expression level of Bax K128R is 1.817264 and the expression level of Bax K190R is 1.982131, which are about twice higher half-life than that of the wild-type Bax. Therefore, it can be confirmed that Bax K128R and Bax K190R have a longer half-life.

### (4) Comparative Analysis of Apoptosis Capability of Wild-Type Bax and Bax Variants

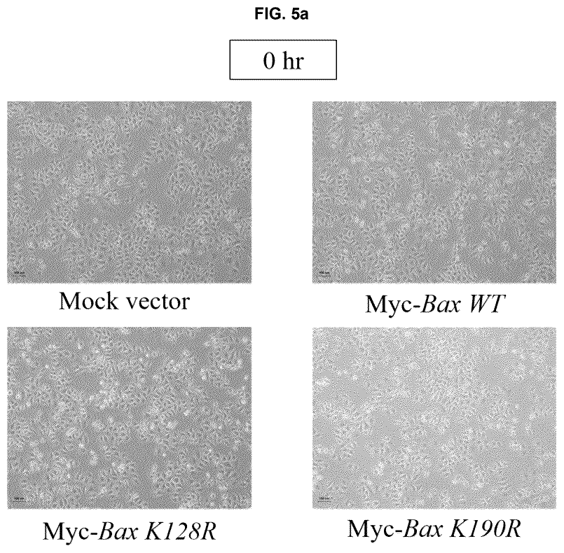

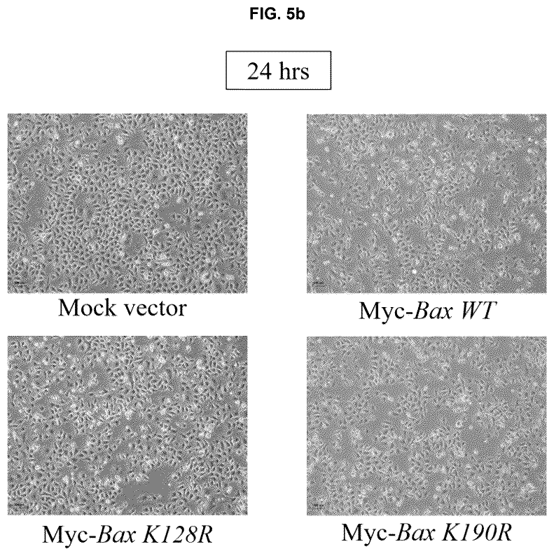

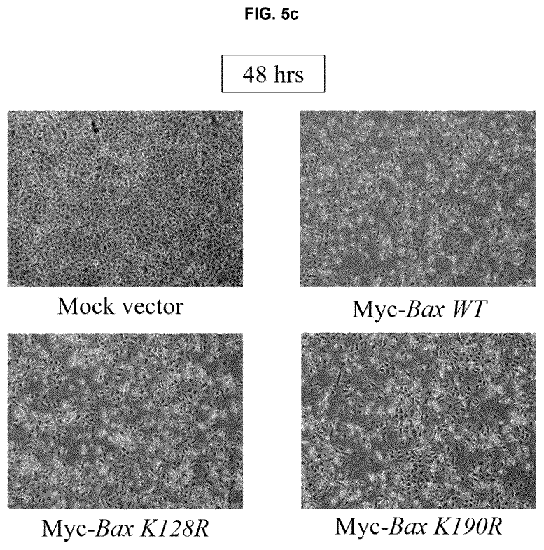

FIGS. 5a to 5c show the results by observing the cells at 0, 24, and 48 hours, after transfecting HeLa cells with a mock vector, wild-type Bax, Bax K128R, and Bax K190R, respectively. It can be confirmed that, when Bax, which has an apoptotic function, was transfected, the number of cells decreased over time compared to the mock vector control group. And, it can be confirmed that apoptosis was observed in the test groups transfected with Bax K128R and Bax K190R in which lysine sites in the Bax were substituted. Therefore, it can be seen that the Bax variants according to the present invention possess the apoptosis capability unique to Bax.

1. A Bax protein variant in which lysine at position 128; lysine at position 190; or lysines at positions 128 and 190 in the Bax protein consisting of the amino acid sequence of SEQ ID NO: 1 is (are) substituted with arginine.

2. The Bax protein variant according to claim 1, consisting of the amino acid sequence of SEQ ID NO: 3.

3. The Bax protein variant according to claim 1, consisting of the amino acid sequence of SEQ ID NO: 5.

4. The Bax protein variant according to claim 1, consisting of the amino acid sequence of SEQ ID NO: 6.

5. A method for increasing the half-life of a Bax protein, the method comprising substituting lysine at position 128 with arginine; lysine at position 190 with arginine; or lysines at positions 128 and 190 with arginine, in the Bax protein consisting of the amino acid sequence of SEQ ID NO: 1.

6. The method for increasing the half-life of a Bax protein according to claim 5, comprising substituting lysine at position 128 with arginine, in the Bax protein consisting of the amino acid sequence of SEQ ID NO: 1.

7. The method for increasing the half-life of a Bax protein according to claim 5, comprising substituting lysine at position 190 with arginine, in the Bax protein consisting of the amino acid sequence of SEQ ID NO: 1.

8. The method for increasing the half-life of a Bax protein according to claim 5, comprising substituting lysines at positions 128 and 190 with arginine, in the Bax protein consisting of the amino acid sequence of SEQ ID NO: 1.

9. A pharmaceutical composition for preventing or treating a solid cancer, comprising the Bax protein variant according to claim 1 as an active ingredient.

10. The pharmaceutical composition according to claim 9, wherein the solid cancer is gastric cancer, hepatic cancer, lung cancer, breast cancer, ovarian cancer, or colorectal cancer.

11. A method for screening a substance having an anticancer activity, comprising
(i) treating cells expressing both a Bax protein variant in which lysine at position 128; lysine at position 190; or lysines at positions 128 and 190 in the Bax protein consisting of the amino acid sequence of SEQ ID NO: 1 is (are) substituted with ar-ginine and a deubiquitinating enzyme USP1, USP7, USP12, or USP49 with a candi-date substance, followed by culturing the cell; and
(ii) measuring whether apoptosis of the cells cultured in step (i) occurred, and then selecting a substance inducing apoptosis of the cells.


SEQUENCE LISTING (14 sequences)

SEQ ID NO: 1 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 2 - DNA, 579 bp, Homo sapiens
  source 1..579: mol_type = genomic DNA
atggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctga

SEQ ID NO: 3 - protein, 192 aa, synthetic construct
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTRVPELIRTIMGWTLDFLRERLLGWIQDQGGWDGLLSYFGTPTWQTVTIFVAGVLTASLTIWKKMG

SEQ ID NO: 4 - protein, 192 aa, synthetic construct
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWRKMG

SEQ ID NO: 5 - protein, 192 aa, synthetic construct
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKRMG

SEQ ID NO: 6 - protein, 192 aa, synthetic construct
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTRVPELIRTIMGWTLDFLRERLLGWIQDQGGWDGLLSYFGTPTWQTVTIFVAGVLTASLTIWKRMG

SEQ ID NO: 7 - DNA, 18 bp, synthetic construct
  source 1..18: mol_type = other DNA
tgcaccagggtgccggaa

SEQ ID NO: 8 - DNA, 18 bp, synthetic construct
  source 1..18: mol_type = other DNA
ttccggcaccctggtgcc

SEQ ID NO: 9 - DNA, 18 bp, synthetic construct
  source 1..18: mol_type = other DNA
atctggaggaagatgggc

SEQ ID NO: 10 - DNA, 18 bp, synthetic construct
  source 1..18: mol_type = other DNA
gcccatcttcctccagat

SEQ ID NO: 11 - DNA, 18 bp, synthetic construct
  source 1..18: mol_type = other DNA
atctggaagaggatgggc

SEQ ID NO: 12 - DNA, 18 bp, synthetic construct
  source 1..18: mol_type = other DNA
gcccatcctcttccagat

SEQ ID NO: 13 - DNA, 18 bp, synthetic construct
  source 1..18: mol_type = other DNA
atggacgggtccggggag

SEQ ID NO: 14 - DNA, 18 bp, synthetic construct
  source 1..18: mol_type = other DNA
tcagcccatcttcttcca